(19) United States
(12) Patent Application Publication
(10) Pub. No.: US 2009/0041755 A1
(43) Pub. Date: Feb. 12, 2009

(54) Methods and Compositions For PDGF-D Activation and Inhibition

(75) Inventor: Ulf Eriksson, Stockholm (SE)

Correspondence Address: CROWELL & MORING LLP;INTELLECTUAL PROPERTY GROUP, P.O. BOX 14300, WASHINGTON, DC, 20044-4300, US

(73) Assignee: Ludwig Institute For Cancer Research, New York, NY (US)

(21) Appl. No.: 11/914,036

(22) PCT Filed: May 10, 2006
(86) PCT No.: PCT/US06/17964

§ 371 Date: Oct. 6, 2008

Related U.S. Application Data
(60) Provisional application No. 60/679,233, filed on May 10, 2005.

(51) Int. Cl. A61K 39/395; C07K 16/00; A61P 35/04

(52) U.S. Cl. 424/130.1; 530/387.1

(57) ABSTRACT

Methods for inhibiting angiogenesis comprising administering urokinase plasminogen activator (uPA) inhibitors, and pharmaceutical compositions suitable for the methods comprising the uPA inhibitors. Also provided are methods for stimulating angiogenesis comprising administering uPA or an agonist thereof to a patient in need thereof, and pharmaceutical compositions comprising an effective amount of uPA or an agonist thereof for the methods of stimulation. The present invention discloses that uPA is a specific PDGF-D activatin rotease.

### FIELD OF THE INVENTION

This invention relates to methods and compositions for activating or inhibiting a platelet-derived growth factor (PDGF), specifically PDGF-D. The invention is based on the discovery that the urokinase plasminogen activator (uPA) is a specific PDGF-D activating protease.

### BACKGROUND OF THE INVENTION

Platelet-derived growth factors (PDGFs) are important for normal tissue growth and maintenance, and are also involved in several pathological conditions such as malignancies, atherosclerosis and fibrosis. PDGF signaling is critical for normal tissue growth and maintenance, and is mediated through two structurally related tyrosine kinase receptors, PDGFR-α and PDGFR-β. The PDGF family consists of disulfide-bonded dimers involving four polypeptide chains: the classical PDGF-A and PDGF-B chains, the newly discovered PDGF-C (Li et al., 2000), and PDGF-D chains (Bergsten et al., 2001; LaRochelle et al., 2001). Unique for PDGF-C and PDGF-D chains are that they share a two-domain organization not found within the classical PDGF chains, with an N-terminal CUB domain in front of the conserved growth factor domain.

PDGF-C and PDGF-D are both secreted from cells as a latent dimer, PDGF-CC and PDGF-DD, respectively, and it is known that regulated proteolytic removal of the CUB domain is required before PDGF-CC and PDGF-DD can bind to and activate their cognate PDGFRs. Activated PDGF-C, like PDGF-A, signals through PDGFR-α homodimers, and activated PDGF-D through PDGFR-β homodimers, whereas PDGF-B binds to and activates both PDGFRs (Heldin and Westermark, 1999; Li and Eriksson, 2003). Other groups have demonstrated that both PDGF-C and PDGF-D are able to activate PDGFRα/β heterodimeric complexes as well (Cao et al., 2002; Gilbertson et al., 2001; LaRochelle et al., 2001). The PDGFs often function in a paracrine mode as they are frequently expressed in cells in close apposition to the PDGFR-expressing mesenchyme (Ataliotis and Mercola, 1997), and the expression of PDGF-C is widespread during embryonic development (Aase et al., 2002; Ding et al., 2000). Relatively little has been published about the tissue distribution of PDGF-D, but some Northern hybridization data are found in Bergsten et al., 2001.

In tumor cells and in cell lines grown in vitro, co-expression of PDGFs and their receptors may also generate autocrine loops resulting in cellular transformation (Betsholtz et al., 1984; Bishop et al., 1998; Keating and Williams, 1988). For the novel PDGFs, PDGF-C and PDGF-D, the PDGF receptor-mediated signaling is further complicated by the requirement for proteolytic activation of the latent factors.

PDGF-C and PDGF-D have been reported to be potent transforming growth factors, however some discrepancies between the reported transforming abilities emphasize the importance in understanding the proteolysis underlying the activation of PDGF-C and PDGF-D (LaRochelle et al., 2002; Li et al., 2003; Zwerner and May, 2001).

In order to understand the physiological roles of PDGF-D mediated signal transduction in these processes, it is important to understand how latent full-length PDGF-DD becomes proteolytically activated to generate a receptor agonist. It was previously shown that the relatively non-specific protease plasmin can be used to activate both PDGF-CC and PDGF-DD from their latent precursors (Bergsten et al., 2001; Li et al., 2000); however, given the wide substrate specificity of plasmin, this protease is unlikely to be a physiologically relevant protease in activation of the novel PDGFs.

The present inventors have shown previously that tissue plasminogen activator (tPA) is a specific activator of PDGF-CC. See e.g. U.S. patent application Ser. No. 10/971,705, which is herein incorporated in its entirety. No specific protease activator for PDGF-DD has been yet identified. Elucidating the identity, localization, and regulation of this protease(s) will greatly enhance understanding of PDGF regulation in vivo. In addition, the role of the CUB domain has not been fully understood. Thus there is a need for elucidating the roles the CUB domain plays in vivo and the identity of the protease(s) involved in PDGF-D activation in vivo.

### SUMMARY OF THE INVENTION

The invention is based on the surprising discovery that urokinase plasminogen activator (uPA) cleaves and activates latent dimeric PDGF-DD. This is a novel role for uPA. uPA is a serine protease with systemic activity. It is expressed as a precursor, pro-uPA which has a molecular weight of about 54 kDa. Pro-uPA is processed into two disulfide-linked chains, A and B, of molecular weights 18 kDa and 33 kDa, respectively. The processing greatly induces the proteolytic activity of uPA. uPA consists of (beginning at the N-terminal end) an EGF-like domain (EGF) (which corresponds to residues 1-45) a kringle domain (which corresponds to residues 46-157), and a trypsin-like protease domain (which corresponds to residues 158-411). The EGF and kringle domains make up the amino terminal fragment, which is mitogenic for human keratinocytes. uPA binds, by its EGF-like domain, to a specific membrane receptor (UPAR) expressed on the surface of many cell types, and converts plasminogen to plasmin on the cell surface. The EGF-like domain is often referred to as the growth factor domain (GFD).

According to one aspect, the invention provides a method for inhibiting proteolytic processing of PDGF-D or PDGF-DD in a mammal in need thereof, comprising administering to the mammal an effective amount of uPA inhibitor. Preferably, the uPA inhibitor is an anti-uPA antibody, a PDGF-D CUB domain or a PDGF-DD CUB domain. A uPA inhibitor may also be an other peptide or small molecule uPA inhibitor or antagonist. It may be anti-sense nucleic acid molecules or small interfering RNA molecules (siRNA, or RNAi). The antibodies may be tagged with a cytostatic or cytotoxic drug or the antibody may be administered together with, or before or after a cytostatic or cytotoxic drug such as cisplatin. The drugs may be given together with the uPA antibody as metronomic therapy ie. continuous or frequent treatment with low does of cancer drugs.

In another embodiment, a therapeutic method is provided for tumor or cancer treatment in a mammal, wherein the tumor is lined by or contains endothelial cells, the method comprising inhibiting proteolytic processing of PDGF-D or PDGF-DD in the mammal. Preferably, the method comprises administering to said mammal an effective amount of a uPA inhibitor. Preferred uPA inhibitors include an anti-uPA antibody, a PDGF-D CUB domain or a PDGF-DD CUB domain. A uPA inhibitor may also be an other peptide or small molecule uPA inhibitor or antagonist. It may be anti-sense or RNAi. The antibodies may be tagged with a cytostatic or cytotoxic drug or the antibody may be administered together with, or before or after a cytostatic or cytotoxic drug such as cisplatin. The drugs may be given together with the uPA antibody as metronomic therapy i.e. continuous or frequent treatment with low does of cancer drugs. The method of the present invention is particularly suitable for the treatment of hemangioendothelioma, an angiosarcoma or a lymphangioma, as well as for the treatment of tumors of the brain, breast, prostate, lung, kidney, liver, and soft tissues etc.

The invention also relates to pharmaceutical compositions and therapeutic methods for treating undesired angiogenesis, modulating normal and pathological tissue remodeling, inhibition of tissue fibrosis; inhibition of ventricular remodeling due to impaired heart function, hypertension or heart infarction; inhibition of inflammation and subsequent fibrosis, inhibition of keloid formation in wound healing and following plastic surgery, kidney diseases that may lead to impaired kidney function and filtration capacity; pathological conditions related to diabetes; the method comprising inhibiting proteolytic processing of PDGF-D or PDGF-DD in the mammal. Preferably, the composition comprises a uPA inhibitor, such as an anti-uPA antibody, a PDGF-D CUB domain or a PDGF-DD CUB domain, or other peptide or small molecule uPA inhibitors or antagonists, and the method comprises administering the composition to said mammal an effective amount of the pharmaceutical composition.

The instant invention additionally embraces a method for modulating, e.g. stimulating, angiogenesis, promoting wound healing and tissue repair, tendon and ligament repair or healing in a mammal in need thereof, or modulating recruitment, proliferation, and maturation of stem cells in vivo or ex vivo, the method comprising administering to the mammal an effective amount of a protease, preferably uPA, to promote proteolytic processing of PDGF-D or of PDGF-DD. Alternatively an agonist of uPA that increases the activity of UPA may be used.

In a particularly advantageous embodiment, the present invention provides a method for stimulating physiological or developmental activities mediated by PDGF-D (e.g. angiogenesis) in a mammal in need thereof, the method comprising administering to the mammal an effective amount of a protease to promote proteolytic processing of PDGF-D or of PDGF-DD. A preferred protease is uPA.

Also provided are pharmaceucial compositions for inhibiting proteolytic processing of PDGF-D or PDGF-DD in a mammal in need thereof, which composition comprises an effective amount of uPA inhibitor, and a pharmaceutically suitable excipient. Many protease inhibitors are uPA inhibitors suitable for the present invention. For example, they include naturally occurring serine protease inhibitors, which are usually polypeptides and proteins which have been classified into families primarily on the basis of the disulfide bonding pattern and the sequence homology of the reactive site. Serine protease inhibitors, including the group known as serpins, have been found in microbes, in the tissues and fluids of plants, animals, insects and other organisms. At least nine separate, well-characterized proteins are now identified, which share the ability to inhibit the activity of various proteases. Several of the inhibitors have been grouped together, namely α₁-proteinase inhibitor, antithrombin III, antichymotrypsin, C1-inhibitor, and α₂-antiplasmin. These inhibitors are members of the α₁-proteinase inhibitor class. Others include the protein α₂-macroglobulin, α₁-antitrypsin (AAT) and inter-alpha-trypsin inhibitor. In addition, as disclosed in U.S. Pat. No. 6,001,355, the seed of Erythrina latissima (broad-leafed Erythrina) and other Erythrina species contains two proteinase inhibitors, referred as DE-1 and DE-3. DE-3 has the property of being an enzyme inhibitor of the Kunitz type and of being an inhibitor for trypsin, plasmin and tPA, and may also have uPA inhibiting activities. U.S. Pat. No. 5,973,118 further discloses a recombinant ETI polypeptide which has a specific inhibitory activity for t-PA and t-PA derivatives. Other peptide serine protease inhibitors are disclosed in U.S. Pat. No. 5,157,019. In addition, U.S. Pat. Nos. 5,424,329 and 5,350,748 disclose staurosporine and other small molecule tPA inhibitors. Likewise, U.S. Pat. No. 5,869,455 discloses N-substituted derivatives; U.S. Pat. No. 5,861,380 protease inhibitors-keto and di-keto containing ring systems; U.S. Pat. No. 5,807,829 serine protease inhibitor-tripeptoid analogues; U.S. Pat. No. 5,801,148 serine protease inhibitors-proline analogues; U.S. Pat. No. 5,618,792 substituted heterocyclic compounds useful as inhibitors of serine proteases. These patents and PCT publications and others as listed infra are incorporated herein, in their entirety, by reference. Other equally advantageous molecules, which may be used instead of α₁-antitrypsin or in combination therewith are contemplated such as in WO 98/20034 disclosing serine protease inhibitors from fleas. Without limiting to this single reference one skilled in the art can easily and without undue experimentation adopt compounds such as in WO98/23565 which discloses aminoguanidine and alkoxyguanidine compounds useful for inhibiting serine proteases; WO98/50342 discloses bis-aminomethylcarbonyl compounds useful for treating cysteine and serine protease disorders; WO98/50420 cyclic and other amino acid derivatives useful for thrombin-related diseases; WO 97/21690 D-amino acid containing derivatives; WO 97/10231 ketomethylene group-containing inhibitors of serine and cysteine proteases; WO 97/03679 phosphorous containing inhibitors of serine and cysteine proteases; WO 98/21186 benzothiazo and related heterocyclic inhibitors of serine proteases; WO 98/22619 discloses a combination of inhibitors binding to P site of serine proteases with chelating site of divalent cations; WO 98/22098 a composition which inhibits conversion of pro-enzyme CPP32 subfamily including caspase 3 (CPP32/Yama/Apopain); WO 97/48706 pyrrolo-pyrazine-diones; WO 97133996 human placental bikunin (recombinant) as serine protease inhibitor; WO 98/46597 complex amino acid containing molecule for treating viral infections and conditions disclosed hereinabove. Other compounds having serine protease inhibitory activity are equally suitable and effective, including but not limited to: tetrazole derivatives as disclosed in WO 97/24339; guanidinobenzoic acid derivatives as disclosed in WO 97/37969 and in U.S. Pat. Nos. 4,283,418; 4,843,094; 4,310,533; 4,283,418; 4,224,342; 4,021,472; 5,376,655; 5,247,084; and 5,077,428; phenylsulfonylamide derivatives represented by general formula in WO 97/45402; novel sulfide, sulfoxide and sulfone derivatives represented by general formula in WO 97/49679; novel amidino derivatives represented by general formula in WO 99/41231; other amidinophenol derivatives as disclosed in U.S. Pat. Nos. 5,432,178; 5,622,984; 5,614,555; 5,514,713; 5,110,602; 5,004,612; and 4,889,723 among many others.

Preferably, the pharmaceutical composition comprises an effective amount of uPA inhibitor for tumor or cancer treatment in a mammal.

The present invention further provides a pharmaceutical composition for stimulating angiogenesis in a mammal in need thereof, comprising an effective amount of uPA to promote proteolytic processing of PDGF-D or of PDGF-DD, and a pharmaceutically acceptable excipient.

A pharmaceutical composition of the invention contains uPA or its inhibitors (“active ingredients”), and an appropriate pharmaceutically acceptable carrier. The term “pharmaceutically acceptable carrier” refers to those solid and liquid substances, which do not significantly or adversely affect the therapeutic properties of the peptides. Suitable pharmaceutical carriers are described in Remington's Pharmaceutical Sciences 1990, pp. 1519-1675, Gennaro, A. R., ed., Mack Publishing Company, Easton, Pa. The serine protease inhibitor molecules of the invention can be administered in liposomes or polymers (see, Langer, R. Nature 1998, 392, 5).

The active ingredients may be administered as free chemicals or pharmaceutically acceptable salts thereof. The terms used herein conform to those found in Budavari, Susan (Editor), “The Merck Index” An Encyclopedia of Chemicals, Drugs, and Biologicals; Merck & Co., Inc. The term “pharmaceutically acceptable salt” refers to those acid addition salts or metal complexes which do not significantly or adversely affect the therapeutic properties (e.g. efficacy, toxicity, etc.).

The pharmaceutical compositions of the present invention may be administered to individuals, particularly humans, either intravenously, subcutaneously, intramuscularly, intranasally, orally, topically, transdermally, parenterally, gastrointestinally, transbronchially and transalveolarly. Topical administration is accomplished via a topically applied cream, gel, rinse, etc. containing therapeutically effective amounts of inhibitors of serine proteases. Transdermal administration is accomplished by application of a cream, rinse, gel, etc. capable of allowing the inhibitors of serine proteases to penetrate the skin and enter the blood stream. Parenteral routes of administration include, but are not limited to, direct injection such as intravenous, intramuscular, intraperitoneal or subcutaneous injection. Gastrointestinal routes of administration include, but are not limited to, ingestion and rectal. Transbronchial and transalveolar routes of administration include, but are not limited to, inhalation, either via the mouth or intranasally and direct injection into an airway, such as through a tracheotomy, tracheostomy, or endotracheal tube. In addition, osmotic pumps may be used for administration. The necessary dosage will vary with the particular condition being treated, method of administration and rate of clearance of the molecule from the body.

The compositions may, where appropriate, be conveniently presented in discrete unit dosage forms and may be prepared by any of the methods well known in the art of pharmacy. Pharmaceutical compositions suitable for oral administration may be presented as discrete unit dosage forms such as hard or soft gelatin capsules, cachets or tablets, each containing a predetermined amount of the active ingredient; as a powder or as granules; as a solution, a suspension or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art., e.g., with enteric coatings.

Oral liquid preparations may be in the form of, for example, aqueous or oily suspension, solutions, emulsions, syrups or elixirs, or may be presented as a dry product for constitution with water or another suitable vehicle before use. Such liquid preparations may contain conventional additives such as suspending agents, emulsifying agents, non-aqueous vehicles (which may include edible oils), or preservative.

The compounds may also be formulated for parenteral administration (e.g., by injection, for example, bolus injection or continuous infusion) and may be presented in unit dose form in ampoules, pre-filled syringes, small bolus infusion containers or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

For topical administration to the epidermis, the compounds may be formulated as ointments, creams or lotions, or as the active ingredient of a transdermal patch. Suitable transdermal delivery systems are disclosed, for example, in Fisher et al. (U.S. Pat. No. 4,788,603) or Bawas et al. (U.S. Pat. Nos. 4,931,279, 4,668,504 and 4,713,224). Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredient can also be delivered via iontophoresis, e.g., as disclosed in U.S. Pat. Nos. 4,140,122, 4,383,529, or 4,051,842. At least two types of release are possible in these systems. Release by diffusion occurs when the matrix is non-porous. The pharmaceutically effective compound dissolves in and diffuses through the matrix itself. Release by microporous flow occurs when the pharmaceutically effective compound is transported through a liquid phase in the pores of the matrix.

Compositions suitable for topical administration in the mouth include unit dosage forms such as lozenges comprising active ingredient in a flavored base, usually sucrose and acacia or tragacanth; pastilles comprising the active ingredient in an inert base such as gelatin and glycerin or sucrose and acacia; mucoadherent gels, and mouthwashes comprising the active ingredient in a suitable liquid carrier.

When desired, the above-described compositions can be adapted to provide sustained release of the active ingredient employed, e.g., by combination thereof with certain hydrophilic polymer matrices, e.g., comprising natural gels, synthetic polymer gels or mixtures thereof.

The pharmaceutical compositions according to the invention may also contain other adjuvants such as flavorings, coloring, antimicrobial agents, or preservatives.

The invention particularly relates to antagonists, such as antibodies or small molecules, that target the site of proteolysis in PDGF-D. A peptide sequence, either a monomer or a dimer, which includes the site of PDGF-D proteolysis can be used as an immunogen for generation of antibodies. The antibodies could be polyclonals, monoclonals, or bispecific antibodies recognizing the PDGF-D proteolytic site and another target e.g. PDGF-C proteolytic site. Preferably, the antibodies would be chimerized, humanized or fully human. They could be F(ab)2 fragments, or single chain antibodies or single domain antibodies. Such antibodies and small molecules essentially protect the site of PDGF-D proteolysis by binding to it and thereby preventing uPA binding and subsequent cleavage. The immunogen could also be a fusion protein of the proteolyic site and another immunogen.

A preferred target for the antagonist comprises the specific binding site (RSRK) on PDGF-D by uPA. The RSRK binding site corresponds to amino acids at positions 254-257 of PDGF-D. It is to be understood that any antibody or small molecule which binds to any 4 or 5 consecutive amino acids within a range of about 15 amino acids upstream and about 15 amino acids down stream of this uPA-binding site of PDGF-D could function as an effective antagonist to prevent proteolytic cleavage of PDGF-D. For example, an antibody that binds PDGF-D at anywhere in the range of amino acids 239-272 of PDGF-D could function as an effective uPA antagonist.

Small molecule screening could use a library of PDGF-D fragments as substrate or the full-length PDGF-D. It is also within the scope of the invention to screen antibodies and small molecules for agonistic effects, i.e., as promoters of proteolysis.

The invention also relates to a molecule comprising a PDGF-D CUB domain or analog which functions as an inhibitor of PDGF-D proteolysis. Such CUB domain molecules (including allelic variants and hybridizing sequences) bind uPA so that the uPA is sequestered away from the full length PDGF-D and thus cannot bring about the proteolytic cleavage of the full length PDGF-D protein.

Another aspect of the invention relates to combined antagonism of proteolysis and inhibition of downstream signaling from the receptor. Blocking proteolysis of the full length PDGF-D prevents formation of the processed or mature form of PDGF-D which binds to the PDGFR-β and thereby inhibits downstream signaling.

In addition, the invention also relates to antagonists for “hemi-dimers” which comprise dimers formed between an unprocessed, full length PDGF-D molecule and a processed, mature form of the molecule, and to a method for inhibiting the activity of such hemi-dimers comprising administering a suitable antagonist.

Antibodies used in the invention are preferably chimeric or humanized or fully human antibodies. The antagonists useful in the invention also may include various fragments of immunoglobulin or antibodies known in the art, i.e., Fab, Fab₂, F(ab′)₂, Fv, Fc, Fd, scFvs, etc. A Fab fragment is a multimeric protein consisting of the immunologically active portions of an immunoglobulin heavy chain variable region and an immunoglobulin light chain variable region, covalently coupled together and capable of specifically binding to an antigen. Fab fragments are generated via proteolytic cleavage (with, for example, papain) of an intact immunoglobulin molecule. A Fab₂ fragment comprises two joined Fab fragments. When these two fragments are joined by the immunoglobulin hinge region, a F(ab′)₂ fragment results. An Fv fragment is a multimeric protein consisting of the immunologically active portions of an immunoglobulin heavy chain variable region and an immunoglobulin light chain variable region covalently coupled together and capable of specifically binding to an antigen. A fragment could also be a single chain polypeptide containing only one light chain variable region, or a fragment thereof that contains the three CDRs of the light chain variable region, without an associated heavy chain moiety or, a single chain polypeptides containing only one heavy chain variable region, or a fragment thereof containing the three CDRs of the heavy chain variable region, without an associated light chain moiety; and multi specific antibodies formed from antibody fragments, this has for example been described in U.S. Pat. No 6,248,516. Fv fragments or single region (domain) fragments are typically generated by expression in host cell lines of the relevant identified regions. These and other immunoglobulin or antibody fragments are within the scope of the invention and are described in standard immunology textbooks such as Paul, Fundamental Immunology or Janeway et al. Immunobiology (cited above). Molecular biology now allows direct synthesis (via expression in cells or chemically) of these fragments, as well as synthesis of combinations thereof. A fragment of an antinody or immunoglobulin can also have bispecific function as described below.

The antagonists may also be bispecific antibodies, which are monoclonal, preferably human or humanized, antibodies that have binding specificities for at least two different antigens. In the present case, one of the binding specificities is for uPA and the other one is for any other antigen, and preferably for a cell-surface protein or receptor or receptor subunit. Methods for making bispecific antibodies are known in the art. Traditionally, the recombinant production of bispecific antibodies is based on the co-expression of two immunoglobulin heavy-chain/light-chain pairs, where the two heavy chains have different specificities [Milstein and Cuello, Nature, 305:537-539 (1983)]. It is also well known within the art of how to generate bispecific antibodies, or bispecific antibody fragments, by using recombinant DNA techniques (Kriangkum et al. Biomol Eng. 2001 September;18(2):31-40).

Suitable antagonists thus may comprise an antibody, an Fv fragment, an F_{c} fragment, an F_{d} fragment, a Fab fragment, a Fab′ fragment, a F(ab)₂ fragment, F(ab′)₂ fragment, an scFvs fragment, a single chain antibody, a multimeric antibody, or any combination thereof. If desired, the immunoglobulin molecule may be joined to a reporter or chemotherapeutic molecule, or it may be joined to an additional fragment, and it may be a monomer or a multimeric product. The immunoglobulin molecule may also be made recombinantly, to include all or part of the variable regions and/or CDRs.

The above methods and compositions are especially suitable for use in human treatment.

### BRIEF DESCRIPTION OF THE DRAWINGS

FIG. 1 shows the domain structures of tPA and uPA. Note the similarities in domain structures between the two proteases. (Fn1, fibronectin 1-like domain; EGF, EGF-like domain; Kringle 1 and 2, kringle 1 and 2-like domains; Trypsin, trypsin-like domain. The numbering refers to the respective amino acid number in the two molecules.

FIG. 2 shows that uPA can activate latent PDGF-DD. (upper panel), expression of uPA was verified by immunoblotting, (middle panel) expression of latent PDGF-DD or chimeric PDGF-D/PDGF-C molecules generates 45 kDa species as visualized using specific antibodies (lower panel), using growth factor domain specific antibodies to PDGF-D, released growth factor domain migrating as a 21 kDa species was found only when coexpressing latent PDGF-DD and uPA (Bergsten et al. 2001, Nature Cell Biol. 3(5) 512-6. PDGF-D is a specific protease-activated ligand for the PDGF beta-Receptor). Expressing PDGF-DD in the absence of uPA did not generate this species. Analysis of all the PDGF-D/PDGF-C chimeric molecules revealed that no released growth factor domain could be visualized using specific antibodies. (PD, PDGF-DD; PC/D, CUB domain from PDGF-C and growth factor domain from PDGF-D; PD/C, CUB domain from PDGF-D and growth factor domain from PDGF-C). The latter chimeric molecule was detected using a PDGF-C specific antiserum (described in Li, X., Pontén, A., Aase, K., Karlsson, L., Abramsson, A., Uutela, M., Backström, G., Hellström, M., Boström, H., Li, H., Soriano, P., Betsholtz, C., Heldin, C. H., Alitalo, K., Östman, A., and Eriksson, U. (2000) Nat. Cell. Biol. 2, 302-309).

### DETAILED DESCRIPTION OF PREFERRED EMBODIMENTS

To identify the enzyme responsible for activation of latent PDGF-DD, uPA was cloned into an expression vector, co-transfected with another expression vector encoding full-length PDGF-D, and determined that released growth factor domain of PDGF-DD migrating as a 21 kDa species was found only when latent PDGF-DD and uPA were co expressed.

Cloning of urokinase plasminogen activator (uPA): Total cellular RNA from AG1523 fibroblastic cells was prepared using the guanidinium thiocyanate/acid phenol method. Single-stranded cDNA was synthesized using AMV Reverse Transcriptase (Amersham) and oligo-dT to prime the reaction. Oligonucleotides flanking the 1293 bp coding sequence of uPA were used under standard PCR reactions with cDNA from the fibroblastic cell line AG1523 cells as template. HindIII/XhoI digested product was cloned into the eukaryotic expression vector pcDNA3.1/Zeo(+) (Invitrogen) and the construct was verified by nucleotide sequencing. All primers used were purchased from Invitrogen. Forward primer (including a HindIII site for in-frame cloning) was 5′-GTA GAA GCT TGA CCT CGC CAC CAT GAG AG (SEQ ID NO: 1) and reverse primer was 5′-GTA GOT CGA GTT ACA GAT CCT CTT CTG AGA TGA GTT TTT GTT CGA GGG CCA GGC CAT TCT CTT C (SEQ ID NO: 2) (including an XhoIII site for in-frame cloning and a myc-tag for detection).

In vitro processing of PDGF-DD. To explore the proteolytic activity of uPA on full-length PDGF-DD, the expression vector for uPA was cotransfected (Lipofectamine Plus, Invitrogen) with an expression vector encoding full-length PDGF-D in Cos-1 cells as previously outlined for the analysis of tPA-mediated activation of PDGF-CC. Following the incubation in serum free medium, aliquots were subjected to TCA-precipitation, and precipitated proteins were analyzed by SDS-PAGE under reducing conditions. Immunoblotting with rabbit polyclonal antibodies against human PDGF-D was used to detect PDGF-D species (Fredriksson, L., Li, II., Fieber, C., Li, X., and Eriksson, U. (2004) EMBO J. 23, 3793-3802; see also Bergeten et al., 2001, supra). Donkey anti-rabbit IgG-HRP linked whole antibody (Amersham Biosciences) was used as secondary antibody. The membranes were subsequently stripped and reprobed with goat anti-human uPA IgG (American Diagnostica Inc.) in order to confirm the presence of uPA. Donkey anti-goat IgG-HRP (sc-2020, Santa Cruz Biotechnology) was used as secondary antibody.

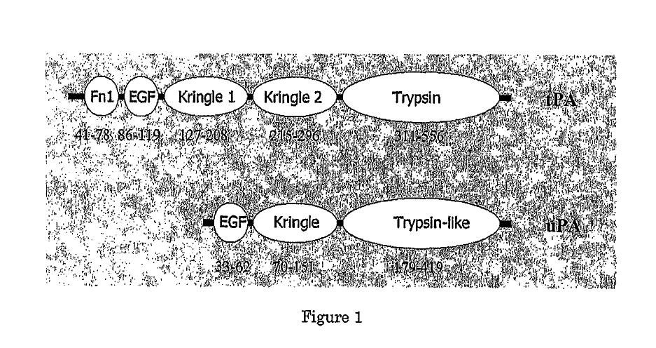

It is known that tPA activates latent PDGF-CC (Fredriksson et al. (2004) EMBO J. 23, 3793-3802). PDGF-CC and PDGF-DD share structural similarities, and uPA also has certain structural similarities to tPA (FIG. 1).

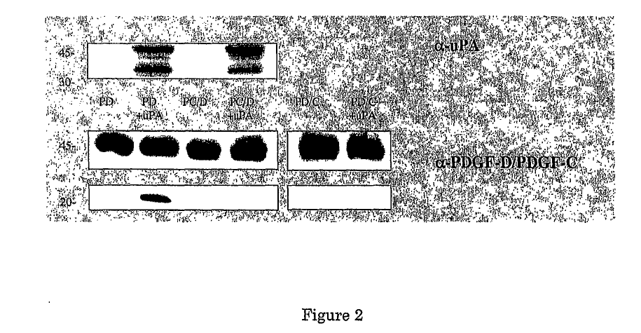

The ability of uPA to activate latent PDGF-DD was investigated using a co-transfection assay similar to the assay previously developed to explore the activation of PDGF-CC using tPA (see e.g. U.S. patent application Ser. No. 10/971,705). SDS-PAGE analysis of PDGF-DD, co-expressed with uPA, revealed that a significant portion of the factor was activated since the released growth factor domain was observed as a distinct 21 kDa species (FIG. 2). In supernatants from cells expressing latent PDGF-DD, but not co-expressing uPA, a released 21 kDa growth factor domain of PDGF-DD was not present. These data show that uPA is able to activate latent PDGF-DD. Given the similar domain structures of the two substrates (PDGF-CC and PDGF-DD) and the two proteases, respectively, the mechanisms of activation of PDGF-CC and PDGF-DD may be very similar. This suggestion was verified using several chimeric PDGF-C/PDGF-D molecules. For the details of how these chimeric factors were generated, see Attachment 1 (Fredriksson et al 2005, J. Bio. Chem.). The results showed that uPA required both the CUB domain and the growth factor domains from PDGF-D to efficiently cleave the substrate (FIG. 2).

uPA is found to be specific for PDGF-DD while tPA is specific for PDGF-CC.

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof. All references cited hereinabove and/or listed below are hereby expressly incorporated by reference.

### REFERENCES

Aase K, Abramsson A, Karlsson L, Betsholtz C, Eriksson U (2002) Expression analysis of PDGF-C in adult and developing mouse tissues. Mech Dev 110: 187-191.
Andrae J, Molander C, Smits A, Funa K, Nister M (2002) Platelet derived growth factor-B and -C and active a-receptors in medulloblastoma cells. Biochem Biophys Res Commun 296: 604-611 Bergsten E, Uutela M, Li X, Pietras K, Oestman A, Heldin C H, Alitalo K, Eriksson U (2001) PDGF-D is a specific, protease-activated ligand for the PDGF b-receptor. Nat Cell Biol 3: 512-516.
Boucher P, Gotthardt M, Li W P, Anderson R G, Herz J (2003) LRP: role in vascular wall integrity and protection from atherosclerosis. Science 300: 329-332.
Bradford M (1976) A rapid and sensitive method for the quantification of microgram quantities of protein utilizing the principle of protein-dye binding. Anal Biochem 72: 248-254.
Cao R, Bra kenhielm E, Li X, Pietras K, Widenfalk J, Oestman A, Eriksson U, Cao Y (2002) Angiogenesis stimulated by PDGF-CC, a novel member in the PDGF family, involves activation of PDGFR-αα and -αβ receptors. FASEB J 16:1575-1583.
Carmeliet P, Schoonjans L, Kieckens L, Ream B, Degen J, Bronson R, De Vos R, van den Oord J J, Collen D, Mulligan R C (1994) Physiological consequences of loss of plasminogen activator gene function in mice. Nature 368: 419-424.
Carroll P M, Tsirka S E, Richards W G, Frohman M A, Strickland S (1994) The mouse tissue plasminogen activator gene 5′ flanking region directs appropriate expression in development and a seizure-enhanced response in the CNS. Development 120: 3173-3183.
Chomczynski P, Sacchi N (1987) Single-step method of RNA isolation by acid guanidinium thiocyanate-phenol-chloroform extraction. Anal Biochem 162: 156-159.
Collen D (2001) Ham-Wasserman lecture: role of the plasminogen system in fibrin-homeostasis and tissue remodeling. Hematology (Am Soc Hematol Educ Program) 1-9.
Deutsch D G, Mertz E T (1970) Plasminogen: purification from human plasma by affinity chromatography. Science 170: 1095-1096.
Dijkmans J, Xu J, Masure S, Dhanaraj S, Gosiewska A, Geesin J, Sprengel J, Harris S, Verhasselt P, Gordon R, Yon J (2002) Characterization of platelet-derived growth factor-C (PDGF-C): expression in normal and tumor cells, biological activity and chromosomal localization. Int J Biochem Cell Biol 34: 414-426.
Ding H, Wu X, Kim I, Tam P P, Koh G Y, Nagy A (2000) The mouse Pdgfc gene: dynamic expression in embryonic tissues during organogenesis. Mech Dev 96: 209-213.
Eghbali M, Tomek R, Woods C, Bhambi B (1991) Cardiac fibroblasts are predisposed to convert into myocyte phenotype: specific effect of transforming growth factor beta. Proc Natl Acad Sci USA 88: 795-799.
Eriksson A, Siegbahn A, Westermark B, Heldin C-H, Claesson-Welsh L (1992) PDGF a- and b-receptors activate unique and common signal transduction pathways. EMBO J 11: 543-550.
Fredriksson, L., Li, H,, Fieber, C., Li, X. and Eriksson, U. (2004) Tissue plasminogen activator is a potent activator of PDGF-CC. EMBO J, 23, 3793-3802.
Gilbertson D G, Duff M E, West J W, Kelly J D, Sheppard P O, Hofstrand P D, Gao Z, Shoemaker K, Bukowski T R, Moore M, Feldhaus A L, Humes J M, Palmer T E, Hart C E (2001) Platelet-derived growth factor C (PDGF-C) a novel growth factor that binds to PDGF α and β receptor. J Biol Chem 276: 27406-27414.
Greenhalgh, D. G., Sprugel, K. H., Murray, M. J., and Ross, R. (1990) Am. J. Pathol. 136, 1235-1246
Heldin C-H, Westermark B (1999) Mechanism of action and in vivo role of platelet-derived growth factor. Physiol Rev 79: 1283-1316 Hoylaerts M, Rijken D C, Lijnen H R, Collen D (1982) Kinetics of the activation of plasminogen by human tissue plasminogen activator—role of fibrin. J Biol Chem 257: 2912-2919.
Kwaan H C (1992) The plasminogen-plasmin system in malignancy. Cancer Metast Rev 11: 291-311.
LaRochelle W J, Jeffers M, McDonald W F, Chillakuru R A, Giese N A, Lokker N A, Sullivan C, Boldog F L, Yang M, Vernet C, Burgess C E, Fernandes E, Deegler L L; Rittman B, Shimkets J, Shimkets R A, Rothberg J M, Lichenstein H S (2001) PDGF-D, a new protease-activated growth factor. Nat Cell Biol 3: 517-521.
Li H, Fredriksson L, Li X, Eriksson U (2003) PDGF-D is a potent transforming and angiogenic growth factor. Oncogene 22: 1501-1510.
Li X, Eriksson U (2003) Novel PDGF family members: PDGF-C and PDGF-D. Cytokine Growth Factor Rev 14: 91-98.
Li X, Pontén A, Aase K, Karlsson L, Abramsson A, Uutela M, Baekstroem G, Hellstro m M, Bostro m H, Li H, Soriano P, Betsholtz C, Heldin C-H, Alitalo K, stman A, Eriksson U (2000) PDGF-C is a new protease-activated ligand for the PDGFa receptor. Nat Cell Biol 2: 302-309.
Lokker N A, Sullivan C M, Hollenbach S J, Israel M A, Giese N A (2002) Platelet-derived growth factor (PDGF) autocrine signaling regulates survival and mitogenic pathways in glioblastoma cells: evidence that the novel PDGF-C and PDGF-D ligands may play a role in the development of brain tumors. Cancer Res 62: 3729-3735.
Lyon et al., 2003, Effect of plasminogen activator inhibitor-1 in diabetes mellitus and cardiovascular disease. Am J Med. 115 Suppl 8A:62S-68S
Nakamura F, Goshima Y (2002) Structural and functional relation of neuropilins. Adv Exp Med Biol 515: 55-69 Nicole O, Docagne F, Ali C, Margaill I, Carmeliet P, MacKenzie E T, Vivien D, Buisson A (2001) The proteolytic activity of tissueplasminogen activator enhances NMDA receptor-mediated signaling. Nat Med 7: 59-64.
Oestman A, Thyberg J, Westermark B, Heldin C-H (1992) PDGF-AA and PDGF-BB biosynthesis: proprotein processing in the Golgi complex and lysosomal degradation of PDGF-BB retained intracellularly. J Cell Biol 118: 509-519.
Ranby M (1982) Studies on the kinetics of plasminogen activation by tissue plasminogen activator. Biochim Biophys Acta 704: 461-469.
Rickles R J, Strickland S (1988) Tissue plasminogen activator mRNA in murine tissues. FEBS Lett 229: 100-106.
Siegfried G, Khatib A M, Benjannet S, Chretien M, Seidah N G (2003) The proteolytic processing of pro-platelet-derived growth factor-A at RRKR(86) by members of the proprotein convertase family is functionally correlated to platelet-derived growth factor-A-induced functions and tumorigenicity. Cancer Res 63: 1458-1463.
Sprugel, K. H., Greenhalgh, D. G., Murray, M. J., and Ross, R (1991) in Clinical and Experimental Approaches to Dermal and Epidermal Repair: Normal and Chronic Wounds (Barbul, A. , et al., eds) , pp. 327-340, Wiley-Liss, Inc., New York
Strickland S (2001) Tissue plasminogen activator in nervous system function and dysfunction. Thromb Haemost 86: 138-143.
Sturzebecher J, Prasa D, Hauptmann J, Vieweg H, Wikstrom P (1997) Synthesis and structure-activity relationships of potent thrombin inhibitors: piperazides of 3-amidinophenylalanine. J Med Chem 40: 3091-3099.
Takeuchi T, Shuman M A, Craik C S (1999) Reverse biochemistry: use of macromolecular protease inhibitors to dissect complex biological processes and identify a membrane-type serine protease in epithelial cancer and normal tissue. Proc Natl Acad Sci USA 96: 11054-11061.
The National Institute of Neurological Disorders and Stroke rtPA Stroke Study Group (1995) Tissue plasminogen activator for acute ischemic stroke. N Engl J Med 333: 1581-1587.
Thielens N M, Bersch B, Hernandez J F, Arlaud G J (1999) Structure and functions of the interaction domains of C1r and C1s: keystones of the architecture of the C1 complex. Immunopharmacology 42: 3-13.
Tsirka S E (2002) Tissue plasminogen activator as a modulator of neuronal survival and function, Biochem Soc Trans 30: 222-225.
Tsirka, S. E., Gualandris, A., Amaral, D. G. and Strickland, S. (1995) Excitotoxininduced neuronal degeneration and seizure are mediated by tissue plasminogen activator. Nature, 377, 340-344.
Uutela M, Lauren J, Bergsten E, Li X, Horelli-Kuitunen N, Eriksson U, Alitalo K (2001) Chromosomal location, exon structure, and vascular expression patterns of the human PDGFC and PDGFD genes. Circulation 103: 2242-2247.
van Hinsbergh, V. W., Kooistra, T., Emeis, J. J. and Koolwijk, P. (1991) Regulation of plasminogen activator production by endothelial cells: role in fibrinolysis and local proteolysis. Int J Radiat Biol, 60, 261-272.
van Zonneveld, A. J., Chang, G. T., van den Berg, J., Kooistra, T., Verheijen, J. H., Pannekoek, H. and Kluft, C. (1986a) Quantification of tissue-type plasminogen activator (t-PA) mRNA in human endothelial-cell cultures by hybridization with a t-PA CDNA probe. Biochem J, 235, 385-390.
van Zonneveld, A. J., Veerman, H. and Pannekoek, H. (1986b) Autonomous functions of structural domains on human tissue-type plasminogen activator. Proc Natl Acad Sci USA, 83, 4670-4674.
Vassalli J D, Sappino A P, Belin D (1991) The plasminogen activator/plasmin system. J Clin Invest 88: 1067-1072.
Wang, Y. F., Tsirka, S. E., Strickland, S., Stieg, P. E., Soriano, S. G. and Lipton, S. A. (1998) Tissue plasminogen activator (tPA) increases neuronal damage after focal cerebral ischemia in wild-type and tPA-deficient mice. Nat Med, 4, 228-231.
Wu Y P, Siao C J, Lu W, Sung T C, Frohman M A, Milev P, Bugge T H, Degen J L, Levine J M, Margolis R U, Tsirka S E (2000) The tissue plasminogen activator (tPA)/plasmin extracellular proteolytic system regulates seizure-induced hippocampal mossy fiber outgrowth through a proteoglycan substrate. J Cell Biol 148: 1295-1304.
Yepes M, Sandkvist M, Coleman T A, Moore E, Wu J Y, Mitola D, Bugge T H, Lawrence D A (2002) Regulation of seizure spreading by neuroserpin and tissue-type plasminogen activator is plasminogen-independent. J Clin Invest 109: 1571-1578.
Yepes M, Sandkvist M, Moore E G, Bugge T H, Strickland D K, Lawrence D A (2003) Tissue-type plasminogen activator induces opening of the blood-brain barrier via the LDL receptor-related protein. J Clin Invest 112: 1533-1540.
Zwerner J P, May W A (2001) PDGF-C is an EWS/FLI induced transforming growth factor in ewing family tumors. Oncogene 20: 626-633.

What is claimed is:

1. A method for inhibiting proteolytic processing of PDGF-D or PDGF-DD in a mammal in need thereof, comprising administering to said mammal an effective amount of a substance which inhibits uPA proteolysis of PDGF-D or PDGF-DD.

2. A method according to claim 1, wherein the substance which inhibits uPA proteolysis of PDGF-D or PDGF-DD is an anti-uPA antibody.

3. A method according to claim 1, wherein the substance which inhibits uPA proteolysis of PDGF-D or PDGF-DD is a PDGF-D CUB domain or a PDGF-DD CUB domain.

4. A method according to claim 1, wherein uPA recognizes a processing site on PDGF-D or PDGF-DD, and wherein the substance which inhibits uPA proteolysis of PDGF-D or PDGF-DD is an antibody against the processing site in PDGF-D or PDGF-DD.

5. A method according to claim 4, wherein the antibody is raised using a polypeptide comprising a partial peptide of at least 15 amino acids encompassing the processing site.

6. A therapeutic method for tumor treatment in a mammal, the method comprising inhibiting proteolytic processing of PDGF-D or PDGF-DD in the mammal.

7. A method according to claim 6, wherein the method comprising administering to said mammal an effective amount of a substance which inhibits PDGF-D proteolysis.

8. A method according to claim 7 wherein the substance which inhibits PDGF-D proteolysis is an anti-uPA antibody.

9. A method according to claim 7, wherein the substance which inhibits PDGF-D proteolysis is a PDGF-D CUB domain or a PDGF-DD CUB domain.

10. A method according to claim 6, wherein uPA recognizes a processing site (RSRK) on PDGF-D or PDGF-DD, and wherein the substance which inhibits uPA proteolysis of PDGF-D or PDGF-DD is an antibody against the processing site.

11. A method according to claim 10, wherein the antibody is raised using a polypeptide comprising a partial peptide of at least 15 amino acids encompassing the processing site.

12. A method according to claim 6, wherein the tumor is lined by or contains endothelial cells.

13. A method according to claim 12, wherein the tumor is a hemangioendothelioma, angiosarcoma, lymphangioma, or a tumor of the brain, breast, lung, prostate, kidney, liver, or soft tissues.

14. A method for treating undesired angiogenesis; modulating normal and pathological tissue remodeling, inhibition of tissue fibrosis; inhibiting ventricular remodeling due to impaired heart function, hypertension or heart infarction; inhibiting inflammation and subsequent fibrosis; inhibiting keloid formation in wound healing and following plastic surgery; treating kidney diseases that may lead to impaired kidney function and filtration capacity; treating pathological conditions related to diabetes; modulating angiogenesis, promoting wound healing and tissue repair, tendon and ligament repair or healing in a mammal in need thereof, or modulating recruitment, proliferation, and maturation of stem cells in vivo or ex vivo, the method comprising inhibiting proteolytic processing of PDGF-D or PDGF-DD in the mammal.

15. A method according to claim 14, wherein the method comprising administering to said mammal an effective amount of a substance which inhibits uPA proteolysis of PDGF-D or PDGF-DD.

16. A method according to claim 15, wherein the substance which inhibits uPA proteolysis of PDGF-D or PDGF-DD is an anti-uPA antibody.

17. A method according to claim 15, wherein the substance which inhibits uPA proteolysis of PDGF-D or PDGF-DD is a PDGF-D CUB domain or a PDGF-DD CUB domain.

18. A method for stimulating angiogenesis in a mammal in need thereof, the method comprising administering to the mammal an effective amount of a protease to promote proteolytic processing of PDGF-D or of PDGF-DD.

19. A method according to claim 18, wherein the protease is uPA.

20. A method according to claim 19, wherein the protease is administered topically.

21. A method according to claim 1, wherein the mammal is a human.

22. A pharmaceutical composition for inhibiting proteolytic processing of PDGF-D or PDGF-DD in a mammal in need thereof, comprising an effective amount of a substance which inhibits proteolytic processing of PDGF-D, and a pharmaceutically suitable excipient.

23. A composition according to claim 22, wherein said substance which inhibits proteolytic processing of PDGF-D comprises a uPA inhibitor.

24. A pharmaceutical composition according to claim 23, wherein the substance which inhibits proteolytic processing of PDGF-D is an anti-uPA antibody.

25. A pharmaceutical composition according to claim 22, wherein the substance which inhibits proteolytic processing of PDGF-D is a PDGF-D CUB domain or a PDGF-DD CUB domain.

26. A pharmaceutical composition for stimulating angiogenesis in a mammal in need thereof, comprising an effective amount of uPA to promote proteolytic processing of PDGF-D or of PDGF-DD, and a pharmaceutically acceptable excipient.

27. A method of inhibiting PDGFR-β receptor signaling, said method comprising administering an effective amount of a substance which inhibits PDGF-D proteolysis.

28. A method according to claim 27, wherein said substance comprises a uPA antagonist.

29. A method according to claim 28, wherein the uPA antagonist is a nucleic acid molecule antisense to a uPA encoding polynucleotide, or an siRNA molecule to a uPA encoding polynucleotide.

30. A method according to claim 28, wherein the antagonist is a small molecule inhibitor of uPA or an antibody against uPA linked with a cytotoxic or cytostatic agent.

31. A method according to claim 28, wherein the uPA antagonist is an anti-uPA antibody.

32. A method according to claim 27, wherein said substance comprises a PDGF-D CUB domain or a PDGF-DD CUB domain.

33. A method for inhibiting the activity of a hemi-dimer formed between an unprocessed, full length PDGF-D molecule and a processed, mature PDGF-D molecule, said method comprising administering an effective amount of a uPA antagonist.

34. A method for stimulating angiogenesis in a mammal in need thereof, the method comprising administering to the mammal an effective amount of a plasminogen activator inhibitor type 1 (PAI-1) antagonist.

35. A method according to claim 34, wherein the PAI-1 antagonist is administered to a site of the mammal topically.

36. An antibody against the uPA processing site on PDGF-D or PDGF-DD, which antibody inhibits activation of PDGF-D or PDGF-DD by uPA.

37. The antibody according to claim 36, wherein the antibody is a monoclonal antibody, a polyclonal antibody, a humanized, chimerized or fully human antibody.

38. A fragment of the antibody according to claim 36, wherein said fragment inhibits activation of PDGF-D or PDGF-DD by uPA, and is a Fab, Fab₂, F(ab′)₂, Fv, Fc, Fd, or scFvs fragment.
